(19) United States
(12) Patent Application Publication
(10) Pub. No.: US 2020/0399189 A1
(43) Pub. Date: Dec. 24, 2020

(54) PROCESS FOR THE PRODUCTION OF SPRINGENE

(71) Applicant: DSM IP ASSETS B.V., Heerlen (NL)

(72) Inventors: Raphael BEUMER, Kaiseraugst (CH); Werner BONRATH, Kaiseraugst (CH); Marc-André MUELLER, Kaiseraugst (CH); Bettina WUESTENBERG, Kaiseraugst (CH)

(21) Appl. No.: 16/976,914

(22) PCT Filed: Mar. 6, 2019
(86) PCT No.: PCT/EP2019/055511

§ 371 Date: Aug. 31, 2020

(30) Foreign Application Priority Data
Mar. 8, 2018 (EP) 18160810.0

(51) Int. Cl. C07C 1/213

(52) U.S. Cl. CPC C07C 1/213; C07C 11/21

(57) ABSTRACT

The application relates to a process for the production of alpha-springene (formula (I)) and beta-springene (formula (II)) from geranyl geranyl acetate (formula (III)).

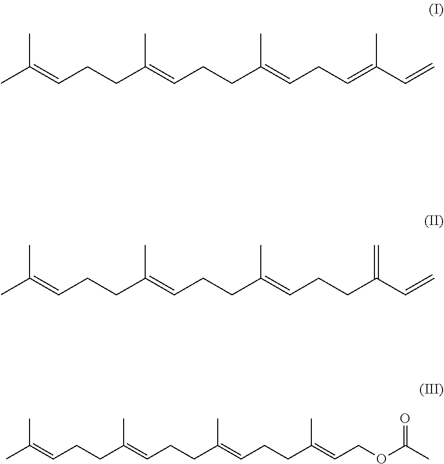

The present invention relates to a new process to produce springene (mixture of α-springene and β-springene).

Springene in the context of the present invention is a mixture of α-springene (compound of formula (I)) and β-springene (compound of formula (II))

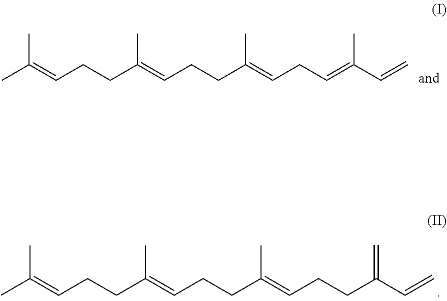

In nature springene can be found in the sex attractant secretion of the springbok (a South African gazelle).

Springene is a very useful compound in the field of isoprenoid chemistry.

Due to the importance of springene, there is always a need for an efficient process of production.

The goal of the present invention was to find an improved synthesis for springene.

It was surprisingly found that springene can be produced from geranyl geranyl acetate (compound of formula (III))

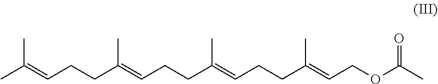 [SMILES: CC(=O)OC/C=C(\C)CC/C=C(\C)CC/C=C(\C)CCC=C(C)C]

in good yields.

The reaction scheme is the following:

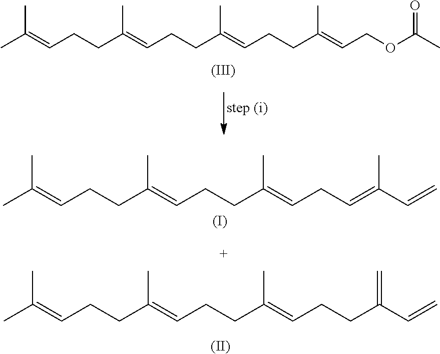

When using geranylgeraniol or geranyllinalool as starting material no conversion was observed for this reaction.

Therefore it is surprising that it worked very well when using geranylgeranyl acetate as starting material.

The starting material (compound of formula (III)) can be obtained commercially or it can be produced by the condensation of the more common natural terpene geraniol with acetic acid.

### Step (i)

In step (i) the compound of formula (III) is used as a starting material.

The reaction of step (i) is usually carried out in at least one solvent.

Suitable solvents are polar aprotic solvents, such as DMSO, carbonates, esters, and ketones.

The reaction of step (i) is usually carried out at elevated temperature (usually above 30° C., usually in range of 30° C.-80° C.).

The reaction of step (i) is usually carried out under an inert gas atmosphere.

The reaction of step (i) is an elimination reaction which is usually and preferably carried out in the presence of a catalyst.

The reaction is performed preferred in presence of a strong heterocylic organic base and a metal catalyst. Useful organic bases are: 1,8-diazabicyclo[5.4.0]undec-7-ene (DBU), 1,5-diazabicyclo[4.3.0]non-5-ene (DBN), Tröger base ((1RS,9RS)-5,13-dimethyl-1,9-diazatetracyclo [7.7.1.0^{2,7}.0^{10,15}]hepta-deca-2(7),3,5,10(15),11,13-hexaene), polyaminophosphazenes (Schwesinger superbases), and 1,4-diazabicyclo[2.2.2]octane (DABCO).

Metal catalysts are preferred from the group of Ni, Pd, and Pt and compound such as Ni(PPh₃)₄, Pd(PPh₃)₄, Pt(PPh₃)₄, Ni(PoTol₃)₄, Pd(PoTol₃)₄, Pt(PoTol₃)₄, Ni(OPPh₃)₄, Pd(OPPh₃)₄, Pt(OPPh₃)₄, Ni(P(C₁₋₆-alkyl)₃)₄, Pd(P(C₁₋₆-alkyl)₃)₄ and Pt(P(C₁₋₆-alkyl)₃)₄, wherein the C₁₋₆-alkyl can be linear or branched.

The obtained product (compounds of formula (I) and (II)) can then be isolated and purified according to known methods. The compounds of formula (I) and (II) can be separated if needed

The mixture, which is obtained by the process according to the present invention comprised usually more of compound of formula (II) than of the compound of formula (I).

A mixture of 10:1 to 2:1 (compound of formula (II) to compound of formula (I)) is obtained

The yield which can be achieved by the process according to the present invention are good.

The obtained product can be used as such or as mentioned above as a building block in the field of terpenoid chemistry.

The following examples serve to illustrate the invention. The temperature is given in ° C. and all percentages are related to the weight.

### EXAMPLES

### Example

### Example 1: Elimination of acetic acid from geranylgeranyl acetate

Under inert gas atmosphere, geranylgeranyl acetate (1.3 mmol) was dissolved in anhydrous DMSO (2.5 ml). With stirring DBU (2 eq.) and Pd(PPh₃)₄ (7 mol %) were added and the reaction mixture was warmed to 60° C. within 15 min. After 3 hours reaction time, the mixture was cooled to room temperature and transferred into a separation funnel with 20 ml of ethyl acetate. The organic phase was washed with water (2×20 ml) and brine (20 ml). The aqueous layers were extracted with ethyl acetate (3×20 ml). The combined organic layers were dried over MgSO₄ and concentrated under reduced pressure. The product was obtained as an isomeric mixture of β- and α-springene (7:3) in 66% yield.

1. Process to produce a mixture of a compound of formula (I) 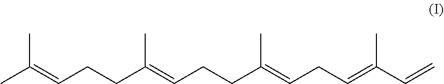 [SMILES: C=C/C(C)=C/C/C=C(\C)CC/C=C(\C)CCC=C(C)C]
and
a compound of formula (II) 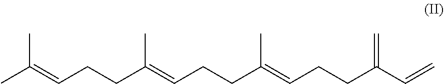 [SMILES: C=CC(=C)CC/C=C(\C)CC/C=C(\C)CCC=C(C)C]
wherein a compound of formula (III) 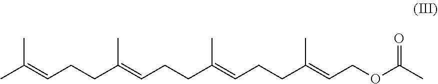 [SMILES: CC(=O)OC/C=C(\C)CC/C=C(\C)CC/C=C(\C)CCC=C(C)C]
is heated in the presence of a catalyst.

2. Process according to claim 1, wherein the reaction is carried out in at least one polar aprotic solvent (preferably DMSO).

3. Process according to claim 1, wherein the reaction of step (i) is carried out at a temperature of 30° C. 120° C.

4. Process according to claim 1, wherein the reaction of step (i) is carried out under an inert gas atmosphere.

5. Process according to claim 1, wherein catalyst is chosen from a strong base chosen from the group consisting of 1,8-diazabicyclo[5.4.0]undec-7-ene (DBU), 1,5-diazabicyclo[4.3.0]non-5-ene (DBN), Tröger base ((1RS,9RS)-5,13-dimethyl-1,9-diazatetracyclo [7.7.1.02,7.010,15]hepta-deca-2(7),3,5,10(15),11,13-hexaene), polyaminophosphazenes (Schwesinger superbases), and 1,4-diazabicyclo[2.2.2]octane (DABCO) and a transition metal complex chosen from the group consisting of Ni(PPh₃)₄, Pd(PPh₃)₄, Pt(PPh₃)₄, Ni(PoTol₃)₄, Pd(PoTol₃)₄, Pt(PoTol₃)₄, Ni(OPPh₃)₄, Pd(OPPh₃)₄, Pt(OPPh₃)₄, Ni(P(C₁₋₆-alkyl)₃)₄, Pd(P(C₁₋₆-alkyl)₃)₄ and Pt(P(C₁₋₆-alkyl)₃)₄, wherein the C₁₋₆-alkyl can be linear or branched.
